(19) United States
(12) Patent Application Publication
(10) Pub. No.: US 2018/0055975 A1
(43) Pub. Date: Mar. 1, 2018

(54) Method for Producing a Dispersion Containing Silver Nanoparticles and Use of a Mixture Containing Silver Nanoparticles as a Coating Agent

(71) Applicant: aap Implantate AG, Berlin (DE)

(72) Inventors: Robert Nusko, Wiesent (DE); Georg Maier, Altenthann (DE); Elvira Dingeldein, Moenchberg (DE); Marco Wolfstaedter, Woerth am Main (DE)

(21) Appl. No.: 15/787,238

(22) Filed: Oct. 18, 2017

Related U.S. Application Data
(63) Continuation of application No. 14/408,467, filed on Dec. 16, 2014, now abandoned.

(30) Foreign Application Priority Data
Dec. 21, 2010 (DE) PCT/DE2010/075165

(51) Int. Cl. A61L 27/54; C09C 1/62; A01N 59/16; A01N 59/20; A61L 27/16; A61L 27/34; A61L 31/10; A61L 31/16; B82Y 30/00; B82Y 40/00

(52) U.S. Cl. CPC A61L 27/54; C01P 2004/64; A01N 59/16; C09C 1/62; A01N 59/20; A61L 27/16; A61L 27/34; A61L 31/10; A61L 31/16; B82Y 30/00; B82Y 40/00; A61L 2420/06; A61L 2430/02; A61L 2300/104; A61L 2300/404; C01P 2002/84; C01P 2004/03; C01P 2004/04; C09C 1/627

(57) ABSTRACT

A method for producing a dispersion containing silver nanoparticles, in particular for producing bone cement or a coating agent for implants from a silver salt.

### FIELD OF THE INVENTION

The invention relates to a coating material comprising a disperse formulation containing silver nanoparticles and methods for production and use thereof, in particular as a coating agent.

### BACKGROUND OF THE INVENTION

Silver is known to have a biocidal effect. Specifically referring to implants in the field of medicine, it is being attempted to an increasing degree to reduce the use of antibiotics or dispense with the use of antibiotics altogether. Silver is an effective alternative in this context.

However, it has been a problem thus far that added silver particles do not achieve a sufficient effect, specifically with bone cements. Presumably, this is related to the specific surface area of the material used in this application usually being too small.

Usually, bone cement is a material that is cured due to a polymerisation reaction. In practical application, for example, methylmethacrylate-based bone cement is known. It usually consists of two components, namely a liquid component and a solid component. The solid component can comprise a mostly fully polymerised bead polymer and a polymerisation initiator and further components, which are usually used to adjust the reaction rate. The monomer component comprises a monomer or a pre-polymer by means of which, after mixing the liquid component and the solid components, a polymerisation reaction is initiated which results in the initially pasty mass being cured to become a solid. Bone cement is used, for example, for endoprostheses, for producing spacers, in multi-part prostheses, and for vertebroplasty and kyphoplasty.

Depending on the application purpose desired, bone cements differing in their stability properties and curing properties can be provided.

To provide an antibiotic effect, it is known to add an antibiotic, such as, for example, gentamicin.

It would be desirable with respect to bone cement and polymer-based coating materials as well to add silver, in addition or alternatively, to attain an antimicrobial effect.

Due to the special properties, in particular the larger specific surface area, it would be desirable, in particular, to add nanoparticulate silver. The addition of nanoparticulate silver to the solid components usually fails simply due to the fact that nanoparticulate silver would be difficult to provide in its solid state, since it agglomerates.

Likewise, adding silver in the liquid phase is difficult since, on the one hand, there are agglomeration effects, and, on the other hand, it has not been possible thus far to provide a sufficiently stable dispersion comprising nanoparticulate silver that stays dispersed also in non-polar liquids or liquids of low polarity such as methylmethacrylate.

According to a general definition, “nanoparticle” is a term referring to particles of a size in the range of less than 100 nm. Accordingly, according to the official definition according to ISO TC 229, the use of the pre-fix “nano” affords a differentiation from particles in the sub-micrometre range (>100 nm). In general, substances referred to as nanomaterial must be presumed to possess changed chemical and physical properties. Accordingly, nanometals of, e.g., gold and silver are of a different colour than the corresponding metals, namely red and yellow, respectively.

Moreover, it is scientifically documented that nanoparticles of a substance possess a higher surface energy. The smaller the particles, the higher is their surface energy. As a result, nanoparticles are usually to be considered to be unstable since they easily react to form new compounds and/or larger, more stable aggregates due to their high surface energy. Referring to nanometals for exemplary purposes, this means that even particles of noble metals are quickly oxidised by atmospheric oxygen as soon as the size of the particles is in the nanometre range.

Accordingly, nanoparticles that are useful for technical applications are obtained only if their surfaces are chemically or physically protected and thus stabilised. Nanoparticles can be called “useful for technical applications” if they maintain or preserve their original particle size from the production through the processing up to their application.

Options for stabilising nanoparticles in dispersions are known from the prior art. There are three essential procedures for the production of metallic nanoparticles. In a first procedure, the nanoparticles are placed on solid supports for stabilisation. The solids are always present at a stable size in the micrometre range in this context. Disadvantages of the use of the products made this way include, on the one hand, the loss of nano-scale and, on the other hand, the high filler load. The filler that is used presently and serves as the basis for the generation of metal nanoparticles comprises grain sizes in the micrometre range and is totally unsuitable, e.g., for producing thin structures or fibres. Moreover, in practical application, the weight fraction of the filler is multiple times larger than the nanometal fraction.

In flame pyrolytic processes, the cluster consisting of micro- and nanoparticles is present as a solid which first needs to be re-dispersed laboriously for further use, which can often no longer be done quantitatively due to influences during storage. Moreover, the distribution of the nanoparticles can never proceed in optimal manner, since it can only be as good as the distribution of the microparticles on which they are deposited.

A second process consists of the synthesis of metal nanoparticles through stabilisation by means of polymers, such as polyvinylpyrrolidone, in the polyol process that has been described widely as standard process in the literature. However, only low metal nanoparticle concentrations are attained in this process (range of less than 0.1 wt.-% silver).

The third variant for generating nanoparticles is a PVD process (physical vapour deposition), in which the metal, on which the process is based, is being evaporated. As before, polymers and/or silicones are used for stabilisation of the nanoparticles thus produced. Generating metal vapour is a very energy-intensive process that requires evacuated process chambers. Accordingly, said production methods are not economical. Moreover, the polymers and silicones used therein cause significant problems during the processing related to the process technology, since re-dispersion is often impossible.

Moreover, it is known from DE 10 2006 056 284 A1 to produce an aqueous dispersion with an antimicrobial effect by mixing an aqueous dispersion of nanoscale particles that contain at least one metal with an antimicrobial effect and an aqueous dispersion of a polymerisation, polycondensation or polyaddition product. The silver nanoparticles are produced through a chemical reduction in water. Sodium chloride is used to stabilise the silver nanoparticles.

One disadvantage shared by all production variants is the poor processability of the metal nanoparticles in melted polymers, such as during the addition of additive to thermoplastic polymers. Solids cannot be incorporated homogeneously without being dispersed first.

Therefore, there continues to be a need for stable dispersions of silver nanoparticles having antimicrobial properties.

### SUMMARY OF THE DISCLOSURE

The invention as characterised in the claims is based on the object to provide stable dispersions of silver nanoparticles, which can be used, in particular, in bone cement or as antibacterial coating for implants and medical devices.

The dispersion is used, in particular, in polymer-based bone cements and coating materials.

The object of the invention is met by a method for producing a dispersion containing silver nanoparticles, by a dispersion containing silver nanoparticles, and by the use of a dispersion containing silver nanoparticles.

Further advantageous details, aspects, and refinements of embodiments of the present invention are evident from the the description, the examples, and the figures.

The invention relates, on the one hand, to a method for producing a dispersion containing silver nanoparticles. Said dispersion is for use, in particular, for producing bone cement or for a coating agent, in particular for implants and medical instruments. Furthermore, a use as antibacterial carrier material both in medicine and for articles of clothing and articles of daily use is envisioned.

According to the invention, a silver salt and a stabiliser are provided.

Furthermore, a reduction agent and an organic polymerisable solvent are provided. Specifically polymers or pre-polymers are provided as organic polymerisable solvent, which can then be used, for example, as coating agent or as component of a bone cement.

A solution is produced from silver salt, stabiliser, and reduction agent.

After production of the solution, a base and an inorganic salt are added.

Upon the addition of the base, nanoparticles precipitate and disperse.

The water remaining in solution is hydrated by means of the inorganic salt. This results in the formation of an aqueous phase, whereas the silver nanoparticles mainly remain in the organic polymerisable solvent due to the stabiliser having been added. The aqueous phase can then be separated, for example by decanting, such that a polymerisable organic solvent comprising silver nanoparticles stays behind.

Accordingly, the invention can provide an essentially anhydrous monomer or pre-polymer containing silver nanoparticles, for example an acrylate, in particular methylmethacrylate or butylacrylate.

It is self-evident that a certain fraction of water can stay behind in the organic solvent since small amounts of water are soluble, for example, in methylmethacrylate.

However, what stays behind is an essentially organic polymerisable solution that can be used, for example, as coating material or as component of a polymerisable material, in particular of bone cement.

The invention also relates to a dispersion containing silver nanoparticles for use, in particular, as bone cement, antibacterial carrier material or coating agent.

The dispersion containing silver nanoparticles comprises silver nanoparticles, at least one stabiliser, and at least one wetting and dispersing additive, whereby the silver nanoparticles are dispersed in a liquid monomer, pre-polymer or polymer.

The invention is based on the insight that having a stabiliser and a further wetting and dispersing additive present allows to provide a dispersion in an organic liquid, for example an acrylate comprising silver nanoparticles, that is stable even over extended periods of time.

Preferably, the stabiliser is selected from the group consisting of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acid ester, polyosypropylene-tri-alkyl acid ester, and mixtures thereof.

Preferably, a non-ionic surfactant, in particular an organo-silicon surfactant, is used as wetting and dispersing additive.

The inventors suspect that nanoparticles are enveloped by the stabiliser and then, as second layer, by the wetting and dispersing agent.

While the stabiliser, in particular in a first production step, serves to ensure that nanoparticles precipitate rather than agglomerate in an aqueous solution, having the wetting and dispersing additive present ensures that the nanoparticles stay dispersed even in an organic liquid of low polarity. The dispersion containing silver nanoparticles can be used, for example, as monomer, in particular for producing bone cement, for a coating solution or as an additive for polymer materials.

Specifically an acrylate or an acrylate precursor, in particular methylmethacrylate is used as polymer in this context.

It is preferable to use, for the present invention, a mixture containing silver nanoparticles, comprising silver nanoparticles and at least one stabiliser selected from the group consisting of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acid ester, and polyoxypropylene-tri-alkyl acid ester.

Said mixture for use for the bone cement according to the invention or the coating agent is described in detail in the following.

All fractions given in units of wt.-% in the present copy shall refer to the weight of the total formulation being equal to 100 wt.-%.

The mixture used presently contains at least one stabiliser selected from the group consisting of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acid ester, and polyoxypropylene-tri-alkyl acid ester. Said stabilisers are compounds having surface-active properties from the group of non-ionic surfactants that are present in liquid form at room temperature. Non-ionic surfactants in the spirit of the invention are surface-active chemical components that comprise uncharged polar and non-polar regions in the same molecule. Moreover, non-ionic surfactants do not comprise any functional groups capable of dissociating.

A mixture according to the invention containing silver nanoparticles contains dispersion-stabilised silver nanoparticles that cannot aggregate into larger agglomerates since the stabilisers used in them are liquid in the temperature range of 0-240° C. In contrast, the prior art includes, for example, many silver nanoparticle products that are supplied as dry powders, but which can be re-dispersed for dispersion in organic solvents, such as methylmethacrylate, only with a high input of mechanical energy, and only incompletely even then, due to their tendency to agglomerate during transport and storage.

Combinations of surface-active components from the classes of chemicals specified above are particularly preferred for use in the present invention. Accordingly, at least two stabilisers are present in the mixture according to a particularly preferred embodiment.

Multiple stabilisers being present can mean different stabilisers from one of the specified classes of chemical compounds or stabilisers from different classes of compounds. Accordingly, in a combination of three different stabilisers, for example three different polyoxyethylene-mono-alkyl acid esters can be used, or, for example, two different polyoxyethylene-mono-alkyl acid esters and one polyoxypropylene-mono-alkyl acid ester or, for example, one polyoxypropylene-di-alkyl acid ester, one polyoxyethylene-tri-alkyl acid ester, and one polyoxypropylene-tri-alkyl acid ester can be used. Any combination of said non-ionic surfactants is feasible.

Particularly preferably, the mixture of stabilisers consists of a combination of non-ionic surfactants from two different classes of the classes of compounds specified above.

According to a particularly preferred embodiment, the stabiliser or stabilisers is or are selected from the group consisting of polyoxyethylene-sorbitan-monolaurate, polyoxyethylene-sorbitan-monopalmitate, polyoxyethylene-sorbitan-monostearate, polyoxyethylene-sorbitan-monooleate, polyoxyethylene-sorbitan-tristearate, polyoxyethylene-glyceryl-trioleate, polyoxyethylene-glyceryl-monolaurate, polyoxyethylene-glyceryl-monooleate, polyoxyethylene-glyceryl-monostearate, polyoxyethylene-glyceryl-monoricinoleate, castor oil, hydrogenated castor oil, and soy bean oil.

In many cases, the stabilisers are not known by their chemicals name, but by their corresponding trade name. In the scope of the present invention, Tween20 ™, Tween40 ™, Tween60 ™, Tween80 ™, Polysorbat™, Tagat TO™, Tagat TO V™, Tagat L2 ™, Tagat S2 ™, Tagat R40 ™, Triton X 100 ™, Hydrogenated Castoroil™, PEG 20 Glycerylstearat™, PEG 20 Glyceryllaurat™, PEG 40 Castoroil™, PEG 25 Glyceryltrioleat™, Newcol™, Montane™, Lonzest™, Liposorb™, Nonion™, Kuplur™, Ionet™, Kemotan™, Grillosan™, Ethylan™, Glycomul™, Emsorb™, Disponil™, Amisol™, Armotan™, Sorbax™, Sorbitan™, Span™, and Tego Pearl™ are preferred stabilisers.

Said list of stabilisers is not comprehensive, since different manufacturers market identical or similar products by different names and/or new non-ionic surfactants of the classes of compounds specified above are synthesised in the future and can also be used in a mixture according to the invention.

If at least two stabilisers are contained in the mixture, these preferably are present in the mixture at a quantitative ratio in the range of 1:1 to 2:1.

Since the non-ionic surfactants present in the mixture act as stabilisers for the nanometal formed, there is a quantitative correlation between the concentrations of the stabiliser and of the metal. According to a further preferred embodiment, the quantitative ratio of silver nanoparticle and stabiliser is in the range of 10:2 up to 10:50, particularly preferably in the range of 10:5 up to 10:10. If multiple stabilisers are present, “quantitative ratio of metal and stabiliser” shall be understood to mean the “quantitative ratio of metal versus the sum of the stabilisers present”. Using the preferred quantitative ratios allows mixtures to be obtained from which particularly stable dispersions of metal nanoparticles can be produced, which can be used universally.

Preferably, the particle size of the metal nanoparticles is 1 to 100 nm, particularly preferably 1 to 50 nm, more particularly preferably 1 to 20 nm.

The present invention also relates to a formulation containing silver nanoparticles comprising a dispersion of any of the mixtures containing metal nanoparticles described above. The formulation according to the invention is liquid and contains no other solid minor components that would limit the options of further use.

The mixture according to the invention as well as the formulation according to the invention contain one or more surface-active components as stabilisers which enable not only the stabilision of the silver nanoparticles, but also the processing (by means of re-dispersion, emulsification) into all other substrates. The technologically most demanding further processing is the processing in thermoplastic materials. Temperatures of up to 300° C. are used in this context. Up to this temperature, it is desirable that the formulation used for adding additive is liquid, which can be realised through the use of one or more surface-active components that are liquid up to a temperature of 300° C., if exposed for a short time. Particularly preferably, at least two stabilisers are present in said formulation. Multiple stabilisers being present can mean different stabilisers from one of the specified classes of chemical compounds or stabilisers from different classes of compounds. Accordingly, in a combination of three different stabilisers, for example three different polyoxyethylene-mono-alkyl acid esters can be used, or, for example, two different polyoxyethylene-mono-alkyl acid esters and one polyoxypropylene-mono-alkyl acid ester or, for example, one polyoxypropylene-di-alkyl acid ester, one polyoxyethylene-tri-alkyl acid ester, and one polyoxypropylene-tri-alkyl acid ester can be used. Any combination of said non-ionic surfactants is feasible.

Particularly preferably, the mixture of stabilisers consists of a combination of non-ionic surfactants from two different classes of the classes of compounds specified above.

Since the at least one non-ionic surfactant is used as stabiliser for the nanometal formed, there is a quantitative correlation between the concentrations of the stabiliser and silver. The formulation according to the invention for use, for example, in methylmethacrylate comprises a quantitative ratio of silver and stabiliser in the range of 10:2 to 10:50. Preferably, the quantitative ratio of metal and stabiliser is 10:5 to 10:20, particularly preferably 10:6 to 10:10. If multiple stabilisers are present, “quantitative ratio of metal and stabiliser” shall be understood to mean the “quantitative ratio of metal versus the sum of the stabilisers present”. Particularly stable dispersions of silver nanoparticles are obtained if the preferred quantitative ratios are used.

Preferably, more than two stabilisers are present in the formulation according to the invention. In this case, the content of a first stabiliser is in the range of 30 to 90 wt.-%, preferably between 40 to 60 wt.-%, particular preferably between 45 to 55 wt.-%. The remaining weight fraction up to 100 percent is accounted for by the further stabilisers used in combination, which in turn account for quantitative fractions of 0 to 100 wt.-%. Accordingly, unlike other specifications given herein, the specifications in wt.-% made presently refer to the total weight of stabilisers as the basis of 100%.

Particularly preferably, one or more stabilisers selected from the group consisting of Tagat TO V™, Tween20™, Tween80™, and Tagat L2™ are present in the formulation.

Using said stabilisers, particularly stable and universally useful dispersions are obtained.

According to an even more particularly preferred embodiment of the present invention, a mixture of Tagat TO V™ and Tween20™ is present as stabiliser in the formulation. Formulations, in which the quantitative ratio of Tagat TO V™ and Tween20™ is in the range of 1:2 up to 2:1 are specifically preferred, and formulations, in which the quantitative ratio of Tagat TO V™ and Tween20™ is approx. 1:1 are particularly preferred.

Referring to particle size, reference shall be made again to the definition given above according to which the silver nanoparticles comprise a particle size of less than 100 nm. In the formulation according to the invention, the particle size of the silver nanoparticles is 1 to 100 nm, preferably 1 to 50 nm, particularly preferably 1 to 20 nm. In this context, the morphology of the silver nanoparticles can take the shape of triangles, cubes, spheres, rods or small plates.

Preferably, the formulation contains stable nano-scale metal particles at a concentration of 0.5 to 60 wt.-%, whereby the quantitative ratio of silver nanoparticle and stabiliser is in the range of 10:2 up to 10:50, preferably in the range of 10:5 up to 10:10. In the preferred ranges, one obtains particularly stable dispersions of silver nanoparticles that can be used universally.

Particularly preferably, the fraction of silver nanoparticles present in the formulation is 1 to 40 wt.-%, preferably the fraction is 5 to 30 wt.-%.

Basically, the formulation according to the invention can be produced using any type of solvent, but it is particularly preferred to use water as solvent such that a disperse aqueous formulation containing silver nanoparticles is produced. The formulation particularly preferably contains at least 70 wt.-% water.

As an alternative to water, an organic solvent can be used to produce the dispersion. Accordingly, this would then be a dispersion in an organic solvent of any of the mixtures containing silver nanoparticles described in more detail above. Even more particularly preferably, the organic solvent is methylmethacrylate.

The present invention also comprises a method for producing the disperse formulations containing metal nanoparticles as described above, comprising the steps of providing a metal salt, providing at least one stabiliser selected from the group consisting of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acidester, polyoxypropylene-tri-alkyl acid ester, providing a reducing agent, providing a solvent, producing a solution of metal salt, stabiliser, and reducing agent, adding a base to the solution, whereby the addition of the base takes place continuously over a period of 5 to 48 h in appropriate manner such that the pH value of the formulation is between 0 and 6.

Using the production method according to the invention, a formulation having a very narrow distribution of particle size of the nanoparticles is obtained.

Preferably, water or an organic solvent is used as solvent. Particularly preferably, this concerns an aqueous solution, i.e. a disperse aqueous formulation containing metal nanoparticles is produced.

Preferably, the base is added continuously over a period of 9 to 30 h. By this means, a formulation having a particularly narrow distribution of particle sizes of the nanoparticles is obtained.

The production method according to the invention is a chemical reduction process. Accordingly, the silver particles are produced from the corresponding salts through chemical reduction. In general, any chemical or physical reducing agent can be used to produce the silver nanoparticles according to the invention. In this context, physical reducing agents shall be understood to mean temperature increase or irradiation with light. The use of a chemical reducing agent is advantageous since this allows turnover rates of 100 percent and very high reaction rates to be attained.

It has been evident, surprisingly, that the presence of at least one stabiliser from the group of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acid ester, and polyoxypropylene-tri-alkyl acid ester, allows very strong reducing agents to be used, which leads to an increased reaction rate without attendant risk of forming any major fraction of large, undesired silver particles.

Among chemical reducing agents, those that generate no reaction side products that stay in the reaction mixture, such as the corresponding oxidised form of the corresponding reducing agent, and would lessen the quality of the disperse aqueous formulation containing silver nanoparticles are preferred. Therefore, according to a preferred embodiment, a reducing agent is used that reacts with the metal ions of the metal salt to form elemental metal and otherwise mainly gaseous reaction products.

Reducing agents that can escape from the reaction solution in their oxidised form as a gaseous substance, such as hydrazine hydrate, are particularly preferred. Obviously, the silver nanoparticles according to the invention can just as well be obtained with any other reducing agent.

The production of metals by reduction can be described by a couple of redox equations. The first partial equation is the reduction equation according to which the metal cation from the metal salt is reduced to the elemental metal. The second partial equation describes the corresponding oxidative process of the oxidation of the reducing agent to the corresponding oxidation product, which, ideally, escapes from the reaction solution in its gaseous state. It is common to all reducing agents that one proton is generated per each electron transferred. Said proton contributes to a very large drop in the pH of the overall reaction solution. The decrease in pH is responsible for the reaction to cease, which is not desired. For this reason, a base needs to be added in order to scavenge the protons thus generated, which slow down the overall reaction.

It has been evident, surprisingly, that the type of the base, the concentration of the base, and the addition rate of the base are crucial for the distribution of particle sizes of the nanoparticles in the formulation thus produced.

It is preferable to use ammonia, potassium hydrogencarbonate or sodium hydroxide for the base. Using said bases, particularly stable dispersions having a narrow distribution of the particle sizes of the nanoparticles are obtained.

In this context, the amount of base added needs to be dosed appropriately such that a dispersion with a neutral pH is obtained once the reaction is completed. The pH is then between pH 5 and pH 9.

Bases and/or proton acceptors are defined through their pK_{b} values. The pK_{b} value is the negative common logarithm of the proton concentration in equilibrium and is therefore a measure of the strength of the base.

Bases having a pK_{b} value in the range of −2 to 10.5, preferably 1.5 to 9.1, particularly preferably in the range of 3.5 to 7.5, are well-suited for producing the formulation according to the invention.

Moreover, aside from the strength of the base, the rate of addition into the reaction mixture is crucial for producing the formulation according to the invention. If the addition is too rapid, the particle size spectrum shifts towards larger particles, which are in the micrometre range in an extreme case. But if the addition is too slow, no turnover rates in excess of 90% are obtained since the nanometal already formed elicits catalytically triggers the degradation of reducing agents and, therefore, there is no longer a reaction partner present for generating silver nanoparticles.

Experiments have shown that the base addition rate for a batch size of 50 kg should be in the range of 9 to 30 hours in order to attain the high quality of silver nanoparticles that corresponds to the scope of the invention. The time of base addition decreases accordingly for smaller batch sizes. The addition time cannot be extended upwards at will since the catalytic degradation of the reducing agent by nanometal already formed impairs the overall yield noticeably after 48 h at the latest.

The base addition rate is appropriate such that the pH of the dispersion is maintained between 0 and 6 at all times. A higher pH leads to a rapid reaction and thus to uncontrolled particle growth. A pH that is too low leads to the reaction ceasing and no nanometal being formed any longer.

The formulation according to the invention can be used in a multitude of applications, whereby clearly advantageous properties are attained in a wide variety of applications. The metal nanoparticles of the formulation according to the invention and/or the metal nanoparticles of a formulation produced according to the method according to the invention can be incorporated, in particular, into different substrates to attain antimicrobial activity.

The present invention also relates to a method for producing bone cement or a coating agent for implants or medical instruments through one of the mixtures containing silver nanoparticles described in more detail above, whereby an inorganic salt is added to one of the formulations according to the inventions containing silver nanoparticles described in more detail above or an inorganic salt is added after implementing one of the methods for producing a formulation containing silver nanoparticles as described in more detail above, whereby the inorganic salt comprises at least one element from the fourth or fifth main group of the periodical system of the elements as component of the anion.

Accordingly, the present invention comprises two variants of methods for producing a mixture containing silver nanoparticles, namely a
Method for producing a mixture containing silver nanoparticles, comprising silver nanoparticles and at least one stabiliser selected from the group consisting of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acid ester, and polyoxypropylene-tri-alkyl acid ester, whereby an inorganic salt is added to a formulation containing silver nanoparticles comprising a dispersion of a mixture containing silver nanoparticles, comprising silver nanoparticles and at least one stabiliser selected from the group consisting of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acid ester, and polyoxypropylene-tri-alkyl acid ester, whereby the inorganic salt comprises at least one element from the fourth or fifth main group of the periodical system of the elements as component of the anion,
and a
Method for producing a mixture containing silver nanoparticles, comprising silver nanoparticles and at least one stabiliser selected from the group consisting of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acid ester, and polyoxypropylene-tri-alkyl acid ester, comprising the steps of
   providing a silver salt;
   providing at least one stabiliser selected from the group consisting of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acid ester, polyoxypropylene-tri-alkyl acid ester, and mixtures thereof;
   providing a reducing agent;
   providing a solvent;
   producing a solution of silver salt, stabiliser, and reducing agent;
   adding a base to the solution, whereby the addition of the base takes place continuously over a period of 5 to 48 h in appropriate manner such that the pH value of the formulation is between 0 and 6;
   adding an inorganic salt, whereby the inorganic salt comprises at least one element from the fourth or fifth main group of the periodical system of the elements as component of the anion.

Accordingly, a mixture according to the invention containing nanoparticles can be obtained from a corresponding formulation containing nanoparticles by adding an inorganic salt. It is irrelevant in this context whether the formulation containing nanoparticles was produced by dispersing a mixture containing nanoparticles or whether the formulation containing nanoparticles was obtained by reducing a silver salt in solution.

Specifically referring to the case, in which the mixture containing nanoparticles is produced from a formulation containing nanoparticles that was obtained by reducing a silver salt in solution, adding an inorganic salt is associated with very special advantages. This is the case, because it has been evident, surprisingly, that adding inorganic salts partitions the dispersion obtained through the addition of a base to the solution of silver salt, stabiliser, and reducing agent, into two chemical phases 1 and 2. In this context, phase 1 contains the silver nanoparticles in the liquid stabiliser mixture used presently. The solvent, the inorganic salts, and the side product ammonium nitrate, are present in phase 2, which is the supernatant phase over phase 1. The two phases are then easy to separate by simple means by decanting the upper phase 2. Phase 1 consisting just of the silver nanoparticles and the liquid stabilisers stays behind.

The silver nanoparticles, which are present in the form of a dispersion and usually comprise particle sizes of 1-20 nm and are chemically stabilised, are thus separated from the side product ammonium nitrate, which is present in the dispersion, and the solvent used presently, namely in particular water. The formulations according to the invention containing silver nanoparticles can thus be made useful to additional application fields. Said application fields include applications, in which the solvent that is used and the side product ammonium nitrate have an interfering effect. Specifically in applications involving polar or aprotic solvents, water and ammonium nitrate counteract dispersion of the stabilised silver nanoparticles.

Particularly good results are obtained if the solvent is water and the inorganic salts are water-soluble inorganic salts. In this case, adding water-soluble inorganic salts partitions the dispersion obtained through the addition of a base to the solution of metal salt, stabiliser, and reducing agent, into two chemical phases 1 and 2. Phase 1 again contains the silver nanoparticles in the liquid stabiliser mixture used presently. Water as the solvent, the water-soluble inorganic salts, and the side product ammonium nitrate, are present in phase 2, which is the supernatant phase over phase 1. The two phases can be separated by decanting the upper aqueous phase 2. Phase 1 consisting just of the silver nanoparticles and the liquid stabilisers stays behind.

Any detailed characterisation of suitable salts, needs to consider their cationic and anionic components separately. Salts generally consist of at least one cation and at least one anion. Cations are not expected to undergo an undesired interaction with the formulation containing stabilised metal nanoparticles, since the silver used presently is present either as uncharged metal or as positively charged cation.

If water is used as the solvent, the selection of the suitable combinations of cations/anions in the form of suitable salts is based on the rationale that the phase separation is caused by the utilisation of the hydration capacity of the water by charged ions. In this context, the hydration capacity is expressed in the capacity to form hydrogen bridges. Moreover, it is the basis of the stability of the dispersion in water as the dispersing medium. Thus, there is a certain competition between the stabilisers enveloping the silver nanoparticles and the dissolved side products of the reaction, such as, for example, ammonium nitrate.

The stabilisers according to the invention used presently are non-ionic macromolecules that are connected to the water molecules through weak dipole-dipole interactions only. Accordingly, the rationale of adding salts is to remove the stabilising influence of the hydrogen bridges on the overall dispersion by introducing electrically charged ions, which engage in significantly stronger interactions with the water dipoles. Usually, the degree of interaction with water depends on the ion radius and on the ion charge in that the interaction increases with decreasing ion radii and increasing ion charge. Their solubility in water is also of import in the selection of suitable salts. The formation of interaction forces with the water molecules increases with increasing solubility of the salts in water.

The selection of anions is also limited by the need to prevent undesired interactions with silver cations that are present. Accordingly, this excludes all anions that form poorly soluble compounds with the silver used presently, i.e., e.g., halides, chalcogenides, and the oxygen compounds thereof.

Therefore, it is particularly preferable for the inorganic salt to comprise at least one element from the fifth main group of the periodic system of the elements as component of the anion, whereby the inorganic salt preferably comprises nitrogen as component of the anion.

It is particularly preferred to then decant the phase forming after addition of the inorganic salt, separating it from the mixture containing silver nanoparticles which consists essentially of silver nanoparticles and stabilisers.

The present invention also relates to a method for producing any of the formulations containing silver nanoparticles described in more detail above, comprising the steps of providing any of the mixtures containing silver nanoparticles described in more detail above, providing a solvent, adding the mixture containing silver nanoparticles to the solvent.

Accordingly, the present invention comprises a method for producing a formulation containing silver nanoparticles, comprising a dispersion of a mixture containing silver nanoparticles comprising silver nanoparticles and at least one stabiliser selected from the group consisting of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acid ester, and polyoxypropylene-tri-alkyl acid ester, comprising the steps of providing a mixture containing silver nanoparticles comprising silver nanoparticles and at least one stabiliser selected from the group consisting of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acid ester, and polyoxypropylene-tri-alkyl acid ester, providing a solvent, adding the mixture containing silver nanoparticles to the solvent.

The chemically stabilised silver nanoparticles according to the invention can be wetted and/or dissolved by the solvent without losing the stabiliser shell required for stabilisation.

Preferably, the solvent is water or an organic solvent. All organic, protic, aprotic, polar, and non-polar compounds and/or mixtures thereof can be used.

According to a particularly preferred embodiment of the present invention, at least one wetting and dispersing additive is added in addition. The wetting and dispersing aids provide for wetting and/or dissolution of the silver nanoparticles by the solvent used presently.

Alkylphenolethoxylates, amino-functional polyesters, phosphorus-containing substances such as organically-modified phosphates, phosphonates, polyphosphorus compounds, and alkylphosphonates or a mixture of said compounds are suitable chemical compounds for use as wetting and dispersing additives.

Particularly preferably, the wetting and dispersing additive is an organically-modified phosphate, a phosphonate, a polyphosphorus compound, an alkylphosphonate, a phosphorus compound comprising mixed organic ligands, an oligomer or a polymer comprising phosphate-containing ligands.

Said wetting and dispersing additives are sold by Evonic, BYK Chemie, and Ciba Geigy.

Accordingly, the chemically-stabilised silver nanoparticles having a preferred particle size of 1-20 nm can be incorporated in organic solvents, in particular in methylmethacrylate, by means of wetting and dispersing additives. Said incorporation can take place by means of the simplest stirring or mixing techniques, since the metal nanoparticles do not need to be re-dispersed due to the use of the stabilisers according to the invention. By this means, stable dispersions of, for example, silver nanoparticles having a particle size of preferably less than 20 nm in organic solvents, preferably in methylmethacrylate, of a concentration of 5,000 mg/kg to 50,000 mg/kg silver content are obtained.

The invention specifically relates to a bone cement, an antibacterial carrier material or a coating agent, in particular an acrylate-based coating agent that can be produced using the method described above.

The invention further relates to a bone cement, an antibacterial carrier material or a coating agent for implants or medical devices, which comprises silver nanoparticles.

Specifically, the coating agent is a liquid coating agent, for example an acrylate or silicone. The coating agent can be applied, for example, by dipping (dip-coating).

According to the invention, the nanoparticles are enveloped by at least one first and one second stabiliser and are dispersed in a polymer.

A polymer shall be understood to mean any form of pre-polymer as well as an essentially not-yet-converted monomer solution, which mainly comprises, for example, methylmethacrylate.

The inventors noted that the use of two different stabilisers, in particular the use of two emulsifiers, renders it feasible to provide a stable dispersion that is maintained even in non-polar liquids.

Specifically the formulation described above is suitable as starting material as it is thought to already comprise silver nanoparticles having a shell made of at least one stabiliser.

But even with said formulation, it is not always certain that there are no precipitation or agglomeration phenomena.

However, the inventors noted that appropriate selection of a second stabiliser, thought to become placed about the first stabiliser much like a second shell, renders it feasible to provide a stable dispersion in a non-polar liquid.

Specifically, a non-ionic surfactant, in particular an organo-silicon surfactant, is used for this purpose. A surfactant of this type is available by the trade name of Tego DISPERS 655.

Specifically, at least 0.1, preferably at least 0.2%, of the second stabiliser are added to the mixture, i.e., for example, the monomer component of a bone cement or the coating solution. This aims to minimise the amount of additional chemical substances.

The inventors noted that an amount of less than 1, preferably less than 0.2%, is sufficient to stabilise nanoparticles having a mean particle size between 5 and 50, preferably between 10 and 20 nm.

Thus a dispersion can be provided, in which at least 90, preferably at least 99% of the silver nanoparticles are smaller than 50, preferably smaller than 20 nm.

Preferably, the nanoparticles are essentially spherical in shape, whereby a spherical shape according to the spirit of the invention shall be understood to be a shape, in which the length, width, and height of the particles differ from each other by less than 20%, meaning that they are not, e.g., needle-shaped particles.

Specifically a silver nanoparticle fraction of between 0.5 and 5, preferably between 1 and 3, % by weight in the polymer allows polymer-based coatings or bone cements to be provided which comprise an antimicrobial effect and in which the use of antibiotic is at least reduced or no antibiotic is used altogether.

The present invention also comprises the use of the formulation according to the invention for surface treatment of implants and medical devices. The special advantages attained through this type of use are illustrated in more detail in the following examples.

The present invention comprises, in particular, the use of the formulation according to the invention for producing antimicrobial surfaces. The special advantages attained through this type of use are illustrated in more detail in the following examples.

The present invention also comprises the use of the formulation according to the invention in silicones as coating material. The special advantages attained through this type of use are illustrated in more detail in the following examples.

The present invention also comprises the use of the formulation according to the invention in thermoplastic materials, preferably in polypropylene. The special advantages attained through this type of use are illustrated in more detail in the following examples.

The present invention also comprises the use of the formulation according to the invention in duroplasts, preferably the use for producing PMMA bone cement. The special advantages attained through this type of use are illustrated in more detail in the following examples.

The present invention also comprises the use of the formulation according to the invention for producing PMMA coatings. The special advantages attained through this type of use are illustrated in more detail in the following examples.

### BRIEF DESCRIPTION OF THE DRAWINGS

The following exemplary embodiments are provided for purposes of illustration of the invention and clarifying its advantages. Said exemplary embodiments shall be illustrated in more detail in conjunction with the drawings. It is self-evident that these specifications must not be construed such as to limit the invention. In the figures,

FIG. 1 shows a UV-VIS spectrum of an aqueous solution, diluted 5,000-fold, of a formulation according to the invention.

FIG. 2 shows measured particle sizes and calculated curve of the scanning electron microscope (SEM) datasets.

FIG. 3 shows the transmission electron microscope (TEM) analysis of the silver nanoparticles.

FIG. 4 shows a transmission electron micrographic (TEM) image of an aqueous solution, diluted 5,000-fold, of a formulation according to the invention.

FIG. 5 shows the results of an algae cultivation experiment.

FIG. 6 shows a transmission electron micrographic (TEM) image of a coated fleece/film laminate.

FIG. 7 shows the kinetics of killing of bacteria applied to the coated fleece of FIG. 6 (E. coli.).

FIG. 8 shows a transmission electron micrographic (TEM) image of a polyester master batch comprising 6,500 mg/kg silver.

FIG. 9 shows micro-fibre strands made of PET/PA comprising 200 mg/kg nanosilver.

FIG. 10 shows the elution behaviour and antimicrobial activity of different polyester micro-fibres.

### DETAILED DESCRIPTION

### Example 1

Formulation of Nanosilver with Hydrazine Hydrate, Ammonia, Tagat TO V™, and Tween20

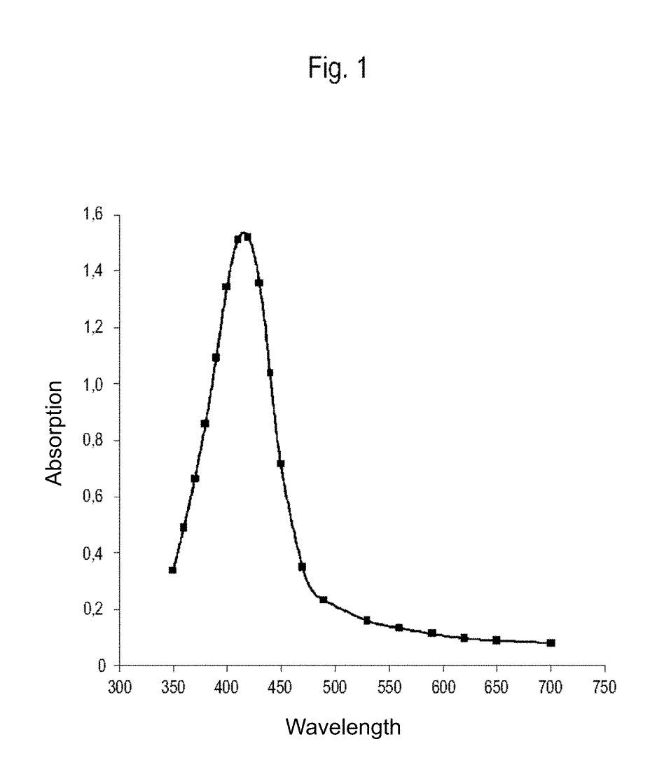

A total of 7,000 g silver nitrate, 1,760 g Tagat TO V™, 1,760 g Tween20™, and 512 g hydrazine hydrate were placed in 28,439 g de-ionised water. The solution was stirred for 3 hours. Then, 5,000 g ammonia solution (14%) were added continuously as droplets over a period of 24 hours. The reaction was complete once the addition was completed and yielded a dispersion having a silver content of 10.0 wt.-%. The particle size and distribution were determined by means of a UV-VIS spectrum (FIG. 1). According to the results, a 10-percent nanosilver dispersion having a nanosilver particle size of 1-30 nm was obtained.

The absorption spectrum was taken on an aqueous solution, diluted 5,000-fold, that contains 20 ppm nanosilver, is clear, and deep-yellow in colour. The UV-VIS spectrum was recorded in the wavelength range of 750 to 350 nm. The absorption values measured showed a peak with a maximum at 410-420 nm and a width at half peak height of approx. 80 nm.

The dispersion properties of the 10-percent dispersion thus obtained were excellent, both in polar and in non-polar solvents, i.e. without any further chemical effort (dispersing aids) or mechanical effort (ultrasound, Ultraturax, etc.), a perfectly clear solution comprising just some colouration due to the plasmonic effect of the silver was obtained.

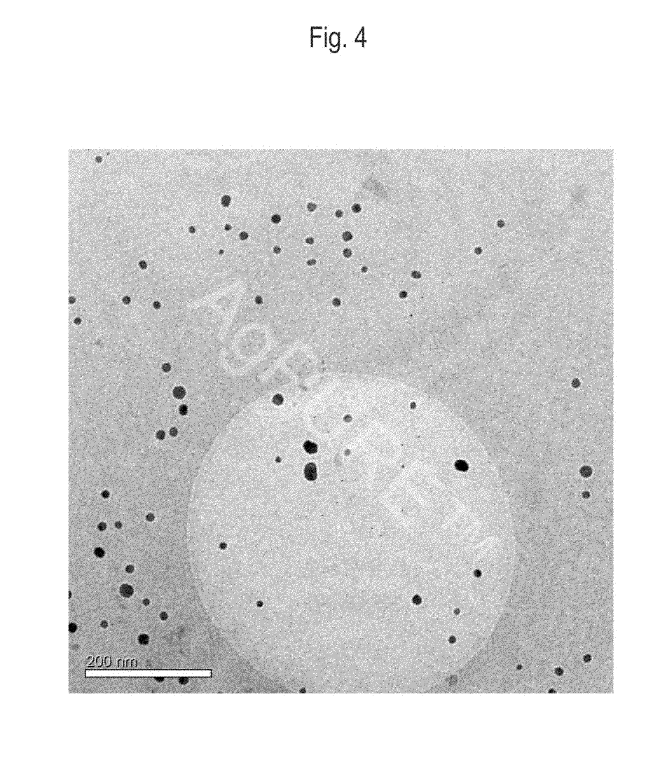

FIG. 4 shows a transmission electron microscopic image (TEM) of the diluted dispersion from example 1. The dark areas visualised in FIG. 4 correspond to the silver nanoparticles that have a particle size of 1-30 nm.

### Example 2

Formulation of Nanosilver with Hydrazine Hydrate, Ammonia, and Tagat TO V™

A total of 7,000 g silver nitrate, 3,520 g Tagat TO V™, and 1,331 g hydrazine hydrate were placed in 27,620 g de-ionised water. The solution was stirred for 3 hours. Then, 5,000 g ammonia solution (14%) were added continuously as droplets over a period of 24 hours. The reaction was complete once the addition was completed and yielded a dispersion having a silver content of 10.0 wt.-%. The particle size and distribution were determined by means of a UV-VIS spectrum. According to the results, a 10-percent nanosilver dispersion having a nanosilver particle size of 1-30 nm was obtained.

### Example 3

Formulation of Nanosilver with Hydrazine Hydrate, Potassium Hydrogencarbonate, Tagat TO V™, and Tween80™

A total of 7,000 g silver nitrate, 2,360 g Tagat TO V™, 1,160 g Tween20 ™, and 1,331 g hydrazine sulfate were placed in 27,620 g de-ionised water. The solution was stirred for 3 hours. Then, 5,000 g potassium hydrogencarbonate solution (1,900 g KHCO₃) were added continuously as droplets over a period of 30 hours. The reaction was complete once the addition was completed and yielded a dispersion having a silver content of 10.0 wt.-%. The particle size and distribution were determined by means of a UV-VIS spectrum (FIG. 1). According to the results, a 10-percent nanosilver dispersion having a nanosilver particle size of 1-30 nm was obtained.

### Example 4

Formulation of Nanosilver with Glucose, Sodium Hydroxide, Tagat L2 ™, and Tween20™

A total of 7,000 g silver nitrate, 2,360 g Tagat L2™, 1,160 g Tween20™, and 3,708 g glucose were placed in 25,243 g de-ionised water. The solution was stirred for 3 hours. Then, 5,000 g sodium hydroxide solution (760 g NaOH) were added continuously as droplets over a period of 30 hours. The reaction was complete once the addition was completed and yielded a dispersion having a silver content of 10.0 wt.-%. The particle size and distribution were determined by means of a UV-VIS spectrum. According to the results, a 10-percent nanosilver dispersion having a nanosilver particle size of 1-30 nm was obtained.

### Example 5

Formulation of Nanocopper with Hydrazine Hydrate, Ammonia, Tagat TO V™, and Tween20™

A total of 10,000 g copper(II) nitrate, 1,760 g Tagat TO V™, 1,760 g Tween20 ™, and 1,090 g hydrazine hydrate were placed in 14,260 g de-ionised water. The solution was stirred for 3 hours. Then, 5,000 g ammonia solution (14%) were added continuously as droplets over a period of 24 hours. The reaction was complete once the addition was completed and yielded a dispersion having a copper content of 10.0 wt.-%.

### Example 6

Processing of the Dispersion from Example 1 for Producing a Liquid, Anhydrous, and Salt-Free Formulation Comprising Silver Nanoparticles

The aqueous dispersion containing silver nanoparticles obtained in Example 1 contained the side product, ammonium nitrate, as an impurity. The silver nanoparticles had particle sizes of 1-20 nm and were chemically stabilised. The silver content was 25 wt.-%. The water content was 366 g and the ammonium nitrate content was 185 g. A total of 1,000 g of said dispersion was placed in a beaker and heated to 45° C. while stirring. The separation of the dispersion into two phases was triggered by adding 78 g potassium nitrate. Once the potassium nitrate was added and dissolved completely, the heating was removed and the stirrer was shut off. Separation of the phases was observed after cool-down. The upper clear aqueous phase 1 was decanted quantitatively. The remaining phase 2 was dark-brown in colour and had syrupy flow properties and a weight of 449 g. The stable dispersion was then ready for incorporation into any organic solvent, in particular methylmethacrylate.

An analysis of the aqueous phase 1 revealed the salt content to be 263 g and the water content to be 366 g.

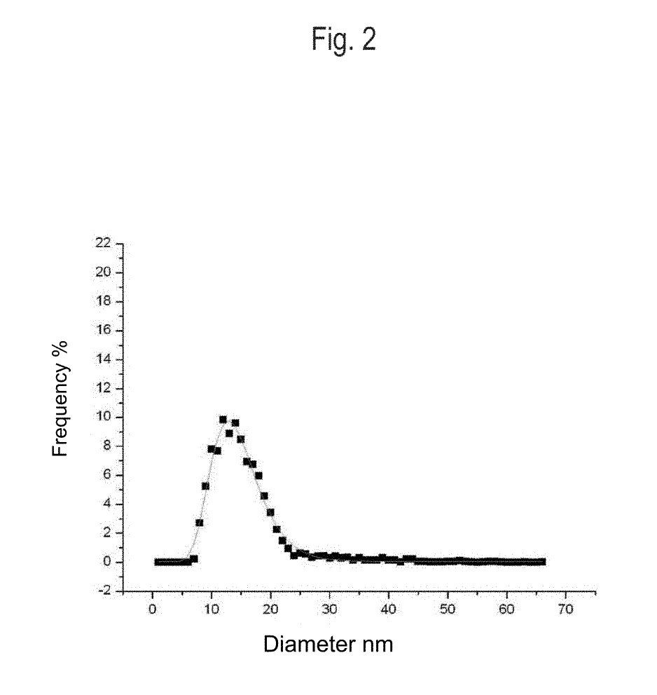

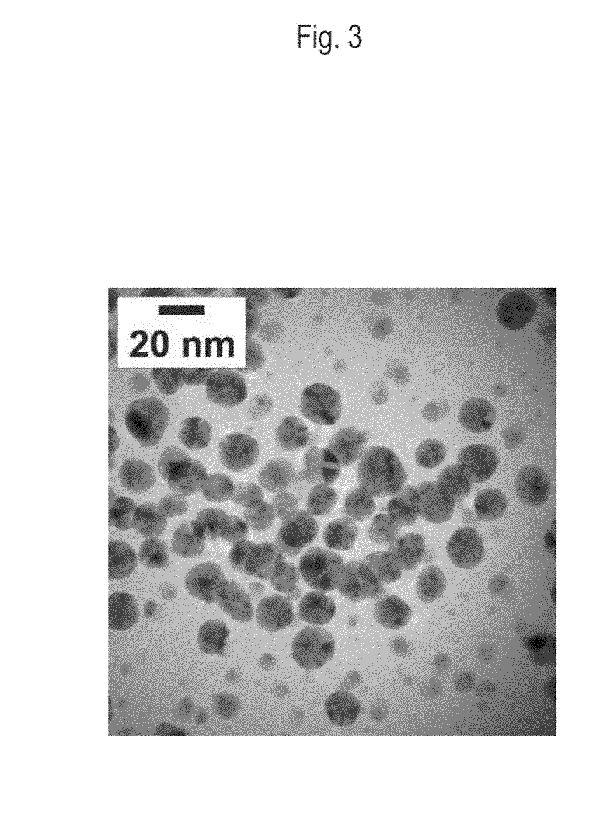

The analysis of the silver-containing phase 2 yielded the following data:
a) The analysis of the total silver content by ashing at 800° C. yielded 250 g silver. With respect to the total formulation, this corresponds to a silver content of 56 wt.-%.
b) The analysis of the particle size distribution of the silver obtained is shown in FIG. 1. This was determined by measuring the UV-VIS absorption spectrum in the wavelength range of 700 nm-350 nm. The peak maximum at 415 nm corresponds to a particle size of 10 nm. The peak width at half-height of maximally 80 nm is a measure of the narrow particle size distribution. Correlation with the SEM (scanning electron microscope) and TEM (transmission electron microscope) analyses, such as shown in FIG. 2 and FIG. 3, allowed the particle size to be specified as D 100<20 nm (100% of the particle diameters are smaller than 20 nm).

### Example 7

Processing of the Dispersion from Example 1 for Producing a Liquid, Anhydrous, and Salt-Free Formulation Comprising Silver Nanoparticles

The aqueous dispersion containing silver nanoparticles obtained in Example 1 contained the side product, ammonium nitrate, as an impurity. The silver nanoparticles had particle sizes of 1-20 nm and were chemically stabilised. The silver content was 10 wt.-%. The water content was 746 g and the ammonium nitrate content was 74 g. The separation of the dispersion into two phases was triggered by adding 202 g potassium nitrate. Once the potassium nitrate was added and dissolved completely, the heating was removed and the stirrer was shut off. Separation of the phases was observed after cool-down. The upper clear aqueous phase 1 was decanted quantitatively. The remaining phase 2 was dark-brown in colour and had syrupy flow properties and a weight of 180 g. The stable dispersion was then ready to be incorporated into any organic solvent, in particular methylmethacrylate.

An analysis of the aqueous phase 1 revealed the salt content to be 276 g and the water content to be 746 g. The analysis of the silver-containing phase 2 yielded the following data:
a) The analysis of the total silver content by ashing at 800° C. yielded 100 g silver. With respect to the total formulation, this corresponds to a silver content of 55 wt.-%.
b) The analysis of the particle size distribution of the silver obtained is shown in FIG. 1. This was determined by measuring the UV-VIS absorption spectrum in the wavelength range of 700 nm-350 nm. The peak maximum at 415 nm corresponds to a particle size of 10 nm. The peak width at half-height of maximally 80 nm is a measure of the narrow particle size distribution. Correlation with the SEM (scanning electron microscope) and TEM (transmission electron microscope) analyses, such as shown in FIG. 2 and FIG. 3, allowed the particle size to be specified as D 100<20 nm (100% of the particle diameters are smaller than 20 nm).

### Example 8

### Use of the Formulation According to the Invention for Treatment of Wood Surfaces

The dispersion according to the invention from Example 1 was incorporated, as known from the prior art, into commercially available linseed oil at a concentration of 100 mg/kg (based on the silver content of the finished product). A stable nanoparticle dispersion was obtained that was well-suited for treating wood surfaces.

The wood fitted with the additive-containing wood oil was resistant to a large variety of chemicals and water. Moreover, the wood surfaces were protected from colonisation by bacteria, i.e. microbes applied to the surfaces described presently die off more rapidly as compared to surfaces bearing no additive-containing wood oil.

### Example 9

### Use of the Formulation According to the Invention for Promoting the Growth of Plants

The dispersion from Example 1 was distributed in water at a concentration of, preferably, 1-100 μg/kg (based on the silver content of the finished product). A stable nanoparticle dispersion that can be used on plants to promote the growth was obtained.

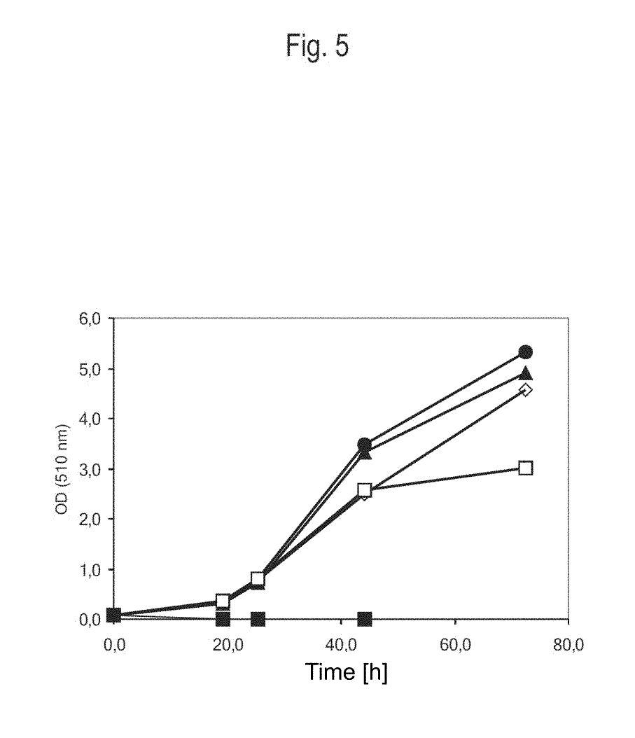

The effect of the aqueous nanosilver dispersion on plant growth was investigated by means of an algae cultivation experiment using Scenedesmus sp. The results of the experiment are shown in FIG. 5. The duration of the experiment after the addition of nanosilver is plotted on the abscissa in FIG. 5. The optical density of the culture at a wavelength of 510 nm, which is proportional to the biomass concentration (algae concentration), is plotted on the ordinate. Cultures without added nanosilver (□: 0 ppb nAg) and cultures of different nanosilver contents (⋄: 1 ppb, : 10 ppb, ▴: 100 ppb, and ▪: 1000 ppb) were investigated for algal growth.

As is evident from FIG. 5, in the absence of added nanosilver, the algae grew exponentially to an OD of approx. 2.5 in the first 42 hours of cultivation. Growth ceases after this time. The OD of the culture with no additive stagnated at approx. 3. The growth curve of the culture containing 1 μg/kg nanosilver additive showed an analogous profile to the culture containing no additive for the first 42 hours of the experiment. Whereas the growth of the culture with no additive nearly ceased between 42 hours and the end of the experiment at 75 hours, the cultures containing 1 μg/g nanosilver additive continue to grow unimpeded during this period and reach a final OD value of 4.7.

As early as after 25 hours, the algae cultures containing 10 μg/kg or 100 μg/kg additive showed markedly increased algal growth as compared to the cultures without additive. The algae grew best at a nanosilver addition of 10 ppb. The final OD value after 75 hours of the experiment was 5.4. Accordingly, the addition of 10 μg/kg nanosilver to Scenedesmus sp. is shown in this experiment to produce a growth increase of 80% after 75 hours of cultivation as compared to an algal culture with no additive. The addition of 1,000 μg/kg nanosilver to the culture had a toxic effect on the algae. The algae added at the start of the experiment die off rapidly.

### Example 10

### Use of the Formulation According to the Invention for the Production of Antimicrobial Surfaces

The dispersion from Example 1 was used for coating at a concentration of, preferably, 5-50 g/kg silver by means of plasma-electrolytic oxidation on metal surfaces, such as, e.g., titanium. Surfaces with a strong antimicrobial activity (R value >3) were obtained.

### Example 11

### Use of the Formulation According to the Invention for the Production of Coatings for Fleece/Film Laminates

The nanosilver dispersion from Example was incorporated into a commercially available fluoropolymer dispersion for coating of films/foils as known from the prior art at a concentration of 150 mg/kg (based on the silver content in the coating). A stable dispersion slightly yellow in colour made of nanosilver particles and fluoropolymer particles was obtained. Said coating was applied with a doctor blade to a PP spunbond/film laminate and dried/fixed/cross-linked by thermal means.

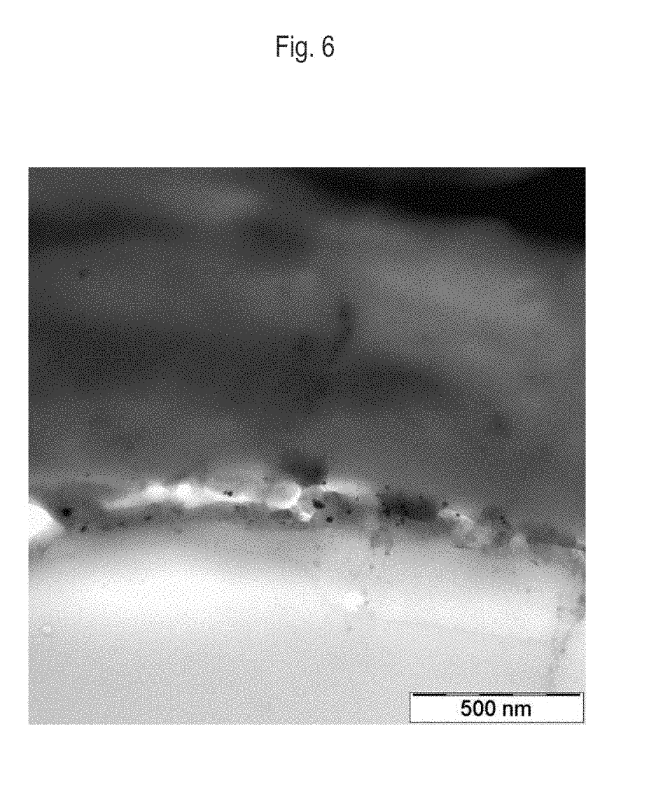

FIG. 6 shows a TEM image of the coating containing nanosilver. The nanoparticles had a typical size of 20 nm (block dots in the TEM image) and were non-aggregated and distributed very homogeneously in the polymer coating.

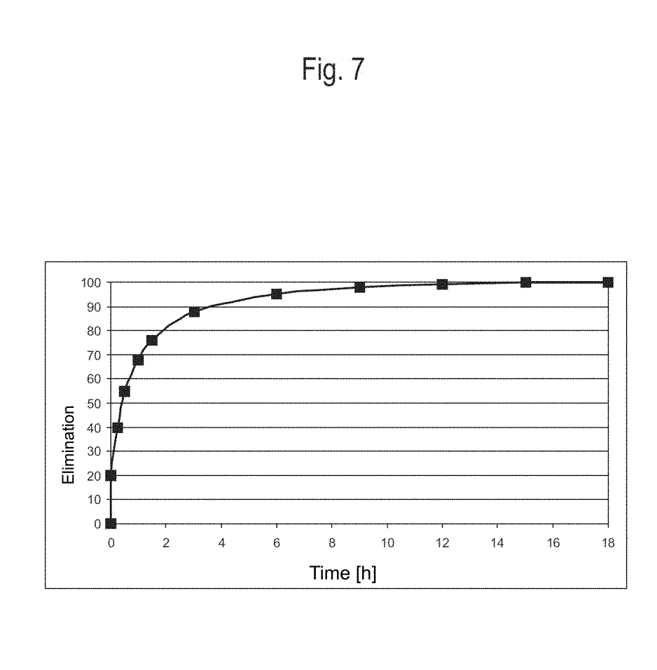

FIG. 7 shows the kinetics of the elimination of bacteria (E. coli.) that had been applied to the coated fleece/film laminate. After as little as 3.5 hours, 90% of the applied pathogens are killed.

Table 1 shows the result of a microbiological test according to JIS 2801 for the fleece/film laminate described above. In the test, 2×10⁵ bacteria each were applied to the nanosilver-coated fleece/film laminate, an uncoated fleece/film laminate, and a standard polystyrene surface. The number of viable germs was determined after a cultivation time of 18 hours. In summary, the number of germs on the nanosilver-coated fleece/film laminate is reduced by 99.8% as compared to standard polystyrene, whereas the uncoated fleece/film laminate showed no germ reduction within the biological variation. Accordingly, the nanosilver-coated fleece/film laminate is considered to have a strong antimicrobial effect.

Table 2 shows the result of the determination of the antimycotic activity of the nanosilver-coated fleece/film laminate. In summary, it can be stated that the nanosilver-coated fleece/film laminate showed significant activity against the 5 fungi tested.

TABLE 1
Microbiological test according to JIS 2801 for
nanosilver-coated fleece/film laminate containing 150 ppm
1. Test result

| | average no. of |
|---|---|
| After 0 h | bacteria [cfu] |
| Inoculum | 2.1 × 10⁵ |

| | average no. of | |
|---|---|---|
| After 18 h | bacteria [cfu] | F value |
| internal standard | 2.8 × 10⁶ | 1.12 |

Duplicate assay of independent samples

| | average no. of | | |
|---|---|---|---|
| After 18 h | bacteria [cfu] | % reduction¹ | R value |
| Fleece/film laminate | 5.1 × 10³ | 99.8% | 2.74 |
| with nanosilver | | | |
| coating | | | |

¹% reduction and R value refer to internal standard

2. Method: JIS Japanese Industrial Standard JIS Z 2801:2000
   Antimicrobial products—Test for antimicrobial activity and efficacy.
   —Plate Count Method—
Test bacteria: Escherichia Coli K12
Modification: sample size: 25 mm×25 mm
   calculation: R value only
   pre-incubation C: LB broth
   pre-incubation D: LB broth
   inoculation medium: 1/500-fold dilution of LB broth (+0.13% Tween 80)
   Incubation: 37° C.
   Sample preparation: UVC-sterilised
      silver in the coating.

TABLE 2
Determination of the antimycotic activity of
nanosilver-coated fleece/film laminate containing 150 ppm
silver in the coating.
1. test result

| Control | Fungal growth | Cat. |
|---|---|---|
| Growth control | strong | 6 |
| Control, sterility | no | |
| Samples | Antimycotic activity | Cat. |
| Cotton (internal | no | 6 |
| standard) | | |
| Fleece/film laminate | significant | 3 |
| with nanosilver | | |
| coating | | |

2. Test method SN 195921 Textile Fabric—Determination of the Antimycotic Activity—modified
Test strains: DSM 40464—Streptomyces abikoensis
   DSM 9122—Scopulariopsis brevicaulis
   DSM 62413—Fusarium solani
   DSM 10640—Penicillium funiculosum
   DSM 2404—Aureobasidium pullulans
Modification: sample size 30 mm diam.
   pre-incubation: potato dextrose agar
   suspension medium: potato dextrose bouillon
   sample inoculation: spraying
   incubation: 25° C., humidity chamber
   incubation time: 40 days
   sample sterilisation: UVC

### Example 12

### Use of the Formulation According to the Invention for the Production of Coatings for Decorative Textile Materials

The nanosilver dispersion from Example 1 was incorporated through means of the prior art into a commercially available fluoropolymer dispersion intended for coating of textile materials at a concentration of 100 mg/kg and 200 mg/kg (based on the silver content of the coating). A stable dispersion slightly yellow in colour made of nanosilver particles and fluoropolymer particles was obtained. Said coating was applied to a decorative textile material and dried/fixed/cross-linked by thermal means.

Table 3 shows the results of microbiological tests according to JIS 1902 on decorative textile materials for floor mats using 2 different hydrophobic aqueous coatings and two nanosilver dosages each. The hydrophobic aqueous coatings AG4 and AG8 are commercially available fluoropolymer coatings. Coating AG4 to which 100 mg/kg or 200 mg/kg nanosilver had been added showed strong germ reduction as compared to an untreated textile material. In the case of coating AG8, the addition of 100 mg/kg nanosilver was insufficient to inhibit germ growth. But the addition of 200 mg/kg effected strong germ reduction as compared to an untreated textile material.

TABLE 3
Microbiological tests according to JIS 1902 on
decorative textile materials for floor mats using 2
different hydrophobic aqueous coatings and two silver
dosages each.
1. Test result

| | average no. of |
|---|---|
| After 0 h | bacteria [cfu] |
| Inoculum | 1.8 × 10⁵ |

| | average no. of | |
|---|---|---|
| After 18 h | bacteria [cfu] | F value |
| Fibre A0 (blank) | 1.5 × 10⁷ | 1.92 |

Duplicate assay of independent samples

| | average no. of | | |
|---|---|---|---|
| After 18 h | bacteria [cfu] | % reduction¹ | R value |
| Control: 080116- | <1.0 × 10² 12 | 99.9993% | 5.18 |
| xhf01 | | | |
| Mesh fabric | | | |
| (Rita), DTY | | | |
| 160/200 F. | | | |
| Floor mat | 1.4 × 10⁴ | 99.9% | 3.03 |
| decorative material | | | |
| Coating | | | |
| AG4 100 ppm | | | |
| Floor mat | 2.4 × 10⁵ | 98.4% | 1.79 |
| decorative material | | | |
| Coating | | | |
| AG4 200 ppm | | | |
| Floor mat | 4.6 × 10⁷ | No | −0.49 |
| decorative material | | | |
| Coating | | | |
| AG8 100 ppm | | | |
| Floor mat | 7.8 × 10³ | 99.9% | 3.29 |
| decorative material | | | |
| Coating | | | |
| AG8 200 ppm | | | |

¹% reduction and R value refer to internal standard

2. Test Method: JIS Japanese Industrial Standard JIS L 1902: 2002 “Testing for antibacterial activity and efficacy on textile products”.
   —Plate Count Method—
Test strain: Escherichia Coli K12
Modification: weight: 0.4 g
   calculation: R value only
   pre-incubation C: LB broth
   pre-incubation D: LB broth
   inoculation medium: phosphate-buffered
   saline plus 0.05% Tween 80
   incubation: 37° C.

### Example 13

### Use of the Formulation According to the Invention in Varnishes and Adhesives

The nanosilver dispersion from Example 1 was incorporated, as known from the prior art, into commercially available varnishes for wood varnishing (in particular stairs and parquet sealing) at a concentration of 270 mg/kg (based on the silver content of the finished product). Stable dispersions containing free nanosilver particles were obtained.

Table 4 shows the results of microbiological tests on water- and solvent-based varnishes each containing 270 ppm nanosilver. The addition of 270 mg/kg nanosilver additive effected strong germ reduction in both varnishes as compared to the standard surface.

TABLE 4
Microbiological tests according to JIS 2801 on
water- and solvent-based varnishes each containing 270 ppm
nanosilver (for stairs and parquet sealing).
1. Test result

| | average no. of |
|---|---|
| After 0 h | bacteria [cfu] |
| Inoculum | 2.1 × 10⁵ |

| | average no. of | |
|---|---|---|
| After 18 h | bacteria [cfu] | F value |
| internal standard | 2.2 × 10⁵ | 0.02 |
| (polystyrene) | | |

Duplicate assay of independent samples

| | average no. of | | |
|---|---|---|---|
| After 18 h | bacteria [cfu] | % reduction¹ | R value |
| Varnish, water- | 1.0 × 10² | 99.95% | 3.35 |
| based containing | | | |
| 270 ppm Ag | | | |
| Varnish, solvent- | 1.0 × 10² | 99.95% | 3.35 |
| based containing | | | |
| 270 ppm Ag | | | |

¹% reduction and R value refer to internal standard

2. Test method: JIS Japanese Industrial Standard JIS Z 2801: 2000
   Antimicrobial products—Test for antimicrobial activity and efficacy.
   —Plate Count Method—
Test strain: Escherichia Coli K12
Modification: sample size: 25 mm×25 mm
   calculation: R value only
   pre-incubation C: LB broth
   pre-incubation D: LB broth
   inoculation medium: 1/500-fold dilution of LB broth (+0.13% Tween 80)
   incubation: 37° C.
   sample preparation: UVC-sterilised

### Example 14

### Use of the Formulation According to the Invention in Silicones

The nanosilver dispersion from Example 1 was incorporated, as known from the prior art, into commercially available silicones at a concentration of 100 mg/kg to 1,000 mg/kg (based on the silver content of the finished product). Stable dispersions containing free nanosilver particles were obtained.

Table 5 shows the results of microbiological tests according to JIS 2802 on 2-component silicones differing in nanosilver content. The addition of just 200 mg/kg nanosilver additive effected strong germ reduction of 97.5% as compared to the standard surface. Strong germ reduction of 99.9% was attained from 500 mg/kg nanosilver.

TABLE 5
Microbiological tests according to JIS 2801 on 2-
component silicones differing in nanosilver content

| | average no. of |
|---|---|
| After 0 h | bacteria [cfu] |
| Inoculum | 1.0 × 10⁵ |

| | average no. of | |
|---|---|---|
| After 18 h | bacteria [cfu] | F value |
| internal standard | 1.4 × 10⁵ | 0.02 |
| (polystyrene) | | |

Duplicate assay of independent samples

| | average no. of | | |
|---|---|---|---|
| After 18 h | bacteria [cfu] | % reduction¹ | R value |
| Silicone | 3.35 × 10³ | 97.5% | 1.6 |
| containing | | | |
| 200 ppm Ag | | | |
| Silicone | <1.0 × 10² | 99.9% | 3.1 |
| containing | | | |
| 500 ppm Ag | | | |
| Silicone | <1.0 × 10² | 99.9% | 3.1 |
| containing | | | |
| 1,000 ppm Ag | | | |

¹% reduction and R value refer to internal standard

2. Test method: JIS Japanese Industrial Standard JIS Z 2801:2000
   Antimicrobial products—Test for antimicrobial activity and efficacy.
   —Plate Count Method—
Test strain: Escherichia Coli K12
Modification: sample size: 25 mm×25 mm
   calculation: R value only
   pre-incubation C: LB broth
   pre-incubation D: LB broth
   inoculation medium: 1/500-fold dilution of LB broth (+0.13% Tween 80)
   incubation: 37° C.
   sample preparation: UVC-sterilised

### Example 15

### Use of the Formulation According to the Invention in Polypropylene Films

The nanosilver dispersion from Example 1 was incorporated, by means of extrusion, into a layer of a multi-layer polypropylene film at typical concentrations of 100 mg/kg to 5,000 mg/kg (based on the silver content of the finished layer). Films with a layer approximately 5 μm in thickness of homogeneously distributed, mainly isolated nanosilver particles were obtained.

Table 6 shows the results of microbiological tests according to JIS 2801 on a multi-layer polypropylene film with different nanosilver contents. The addition of 2,100 mg/kg or 3,200 mg/kg nanosilver additive each effected strong germ reduction of approx. 99% as compared to the standard surface.

TABLE 6
Microbiological tests on a multi-layer polypropylene film.
1. Test result

| | average no. of |
|---|---|
| After 0 h | bacteria [cfu] |
| Inoculum | 1.7 × 10⁵ |

| | average no. of | |
|---|---|---|
| After 18 h | bacteria [cfu] | F value |
| internal | 5.1 × 10⁵ | 0.5 |
| standard | | |
| (polystyrene) | | |

Duplicate assay of independent samples

| | average no. of | | |
|---|---|---|---|
| After 18 h | bacteria [cfu] | % reduction¹ | R value |
| Control, | <1.0 × 10² | >99.98% | >3.7 |
| antibacterial | | | |
| Film comprising | 5.8 × 10³ | 98.87% | 1.9 |
| 2,100 ppm Ag on | | | |
| the internal | | | |
| surface | | | |
| Film comprising | 6.9 × 10³ | 98.66% | 1.9 |
| 3,200 ppm Ag on | | | |
| the internal | | | |
| surface | | | |

¹% reduction and R value refer to internal standard

2. Test method: JIS Japanese Industrial Standard JIS Z 2801:2000
   Antimicrobial products—Test for
   antimicrobial activity and efficacy.
   —Plate Count Method—
Test strain: Escherichia Coli K12
Modification: sample size: 30 mm×40 mm
   calculation: R value only
   pre-incubation: LB medium
   inoculation medium: 1/500-fold dilution of LB broth (+0.13% Tween 80)
   incubation: 37° C.
   sample preparation: no

### Example 16

### Use of the Formulation According to the Invention in Polypropylene Bath Liquor Containers

The nanosilver dispersion from Example 1 was incorporated, by means of extrusion, into a polypropylene film at a concentration of 6,500 mg/(based on the silver content). A master batch containing mostly nanosilver particles that are separate from each other was obtained in this context. The nanosilver master batch was incorporated into polypropylene bath liquor containers at typical concentrations of 100 mg/kg to 5,000 mg/kg (based on the silver content of the finished polymer). The bath liquor containers are intended for accommodation and temporary storage of spent suds and/or washing solutions from dish- and fabric-washing processes. The addition of nanosilver additive was intended to prevent the colonisation of the polymer by germs.

Table 7 shows the results of microbiological tests according to JIS 2801 on polypropylene bath liquor containers of different nanosilver contents. The addition of 520 mg/kg or 1,000 mg/kg or 2,000 mg/kg nanosilver additive each effected strong germ reduction in excess of 99.99% as compared to the standard surface.

TABLE 7
Microbiological tests according to JIS 2801 on
polypropylene bath liquor containers
1. Test result

| | average no. of |
|---|---|
| After 0 h | bacteria [cfu] |
| Inoculum | 3.6 × 10⁵ |

| | average no. of | |
|---|---|---|
| After 18 h | bacteria [cfu] | F value |
| internal standard | 1.1 × 10⁶ | 0.5 |
| (polystyrene) | | |

| | | | |
|---|---|---|---|
| | average no. of | | |
| After 18 h | bacteria [cfu] | % reduction¹ | R value |

Duplicate assay of independent samples

| | | | |
|---|---|---|---|
| Control, | <1.0 × 10² | >99.99% | >4.0 |
| antibacterial | | | |

Triplicate assay of independent samples

| | | | |
|---|---|---|---|
| Bath liquor | <1.0 × 10² | >99.99% | >4.0 |
| container | | | |
| comprising 520 ppm | | | |
| Ag | | | |
| Bath liquor | <1.0 × 10² | >99.99% | >4.0 |
| container | | | |
| comprising 1,000 ppm | | | |
| Ag | | | |
| Bath liquor | <1.0 × 10² | >99.99% | >4.0 |
| container | | | |
| comprising 2,000 ppm | | | |
| Ag | | | |

¹% reduction and R value refer to internal standard

2. Test method: JIS Japanese Industrial Standard JIS Z 2801:2000
   Antimicrobial products—Test for
   antimicrobial activity and efficacy.
   —Plate Count Method—
Test strain: Escherichia Coli K12
Modification: sample size: 30 mm×40 mm
   calculation: R value only
   pre-incubation: LB medium
   inoculation medium: 1/500-fold dilution of LB broth (+0.13% Tween 80)
   incubation: 37° C.
   sample preparation: no

### Example 17

### Use of the Formulation According to the Invention in Wood Plastic Composites (WPC)

The nanosilver dispersion from Example 1 was added to the mixture during the extrusion of PVC and wood flour at a concentration of 50 mg/kg to 1,000 mg/kg. Weatherproof and mildew-resistant WPC materials were obtained.

### Example 18

### Use of the Formulation According to the Invention in Polyolefin Form Body

Aided by white oil, the nanosilver dispersion from Example 1 was mixed with polymer beads made of polyolefins (PE and/or PP) such that a silver concentration of 50 mg/kg to 500 mg/kg was established in the finished mixture. Said mixture was pressed in a press into form bodies (e.g. cutting boards, filters, cosmetics applicators) and reworked by mechanical means. The resulting cutting boards possessed an antimicrobial activity with an R value between 1 and 4, depending on silver content.

### Example 19

Use of the Formulation According to the Invention for the Production of Textile Materials Coated with PVC Plastisol

The nanosilver dispersion from Example 1 was incorporated into PVC Plastisol at typical concentrations of 400 mg/kg (based on the silver content of the finished polymer). The Plastisol was used to coat textile knitwear. This produced mats, which can be used, for example, as floor covering in damp rooms, as gymastics mat, as carpet anti-slip mat or as dish rack. The addition of nanosilver additive was intended to prevent the colonisation of the polymer by microorganisms.

Table 8 shows the results of microbiological tests on polypropylene bath liquor containers to which 400 mg/kg nanosilver additive had been added. The addition of 400 mg/kg nanosilver additive effected a significant antimycotic activity against the 5 fungi tested.

TABLE 8
Test for antimycotic activity of PVC Plastisol
with a silver content of 400 ppm.

| Control | Fungal growth | Cat. |
|---|---|---|
| Cotton (internal | strong | 6 |
| standard) | | |
| Growth control | strong | 6 |
| Control, sterility | no | |
| Sample | Antimycotic activity | Cat. |
| PVC Plastisol | significant | 5 |
| untreated, | | |
| washed 15x | | |
| PVC Plastisol with | no | 3 |
| 400 ppm Ag, | | |
| washed 15x | | |

2. Test method: SN 195921—Textile Fabric—Determination of the Antimycotic Activity—modified
Test strains: DSM 40464—Streptonyces Abikoensis
   DSM 9122—Scopulariopsis Bervicaulis
   DSM 62413—Fusarium Solani
   DSM 10640—Penicillium Funiculosum
   DSM 2404—Aureobasidium Pullulans
Modification: sample size 30 mm diam.
   pre-incubation: potato dextrose agar
   suspension medium: potato dextrose bouillon
   sample inoculation: spraying
   incubation: 25° C.
   incubation time: 41 days
   sample sterilisation: UVC

### Example 20

### Use of the Formulation According to the Invention in PMMA Bone Cement

The nanosilver dispersion from Example 1 was incorporated, by means of the prior art, into commercially available PMMA bone cements at typical concentrations of 100 mg/kg to 5,000 mg/kg (based on the silver content of the finished product). It can be incorporated into either the dry PMMA powder or into the liquid MMA monomer. After curing, bone cements with a homogeneous distribution of mainly isolated nanosilver particles were obtained.

Table 9 shows the results of different elution tests. Accordingly, it is evident from Table 9 that a bone cement test body fitted with 2,149 mg/kg eluted with 10 ml SimulatedBodyFluid (SBF) at an elution temperature of 37° C. for 12 days leaks 4 ng silver per mm² of surface area into the solution. It is evident from the subsequent row in Table 9 that a slight increase of the nanosilver content of the test body to 2,500 mg/kg and reduction of the elution time to 5 days does not change the eluted amount of silver significantly. In an ageing test, a quantity of 13 ng/mm² was eluted by boiling the test body in SBF. Refreshing the elution liquid daily, an equilibrium of 1.2 ng/mm² per day was established.

TABLE 9
Acrylate bone cement (PMMA) containing 2,500 ppm Ag.

| | nAg | | | | |
|---|---|---|---|---|---|
| | content | | | | Eluted |
| | in test | Elution | Elution | Elution | amount of |
| | body | volume | temp. | time | silver |
| Release | 2,149 ppm | 10 mL | 37° C. | 12 d | 4 ng/mm² |
| kinetics | | SBFm | | | (0.4 ng/g |
| | | | | | mm²) |
| Variation | 2,500 ppm | 10 mL | 37° C. | 5 d | 4 ng/mm² |
| of silver | | SBFm | | | (0.4 ng/g * |
| contents | | | | | mm²) |
| Ageing | 2,500 ppm | 10 mL | 100° C. | 5 h | 13 mg/mm² |
| experiment | | SBFm | | | (1.3 mg/g * |
| | | | | | mm²) |
| Replenishment | 2,500 ppm | 1 mL | 37° C. | 33 d | (1.2 ng/g * |
| of elution | | SBFm | | | mm, |
| liquid | | | | | days 5 to |
| | | | | | 10) |

### Example 21

### Use of the Formulation According to the Invention for the Production of PMMA Coatings

The nanosilver dispersion from Example 1 was incorporated, as known from the prior art, into commercially available PMMA preparations at typical concentrations of 100 mg/kg to 5,000 mg/kg (based on the silver content of the finished product). It can be incorporated into either the dry PMMA powder or into the liquid MMA monomer. The ready-mixed preparations were used for coating of mainly medical products. After curing, PMMA coatings with a homogeneous distribution of mainly isolated nanosilver particles were obtained.

### Example 22

### Use of the Formulation According to the Invention for the Production of Synthetic Fibres

The nanosilver dispersion from Example 1 was incorporated, by means of extrusion, into commercially available thermoplasts, such as, e.g., polypropylene, polyester, polyamide, at typical concentrations of 1,000 mg/kg to 20,000 mg/kg (based on the silver content). Masterbatches containing mostly nanosilver particles that are separate from each other were obtained in this context.

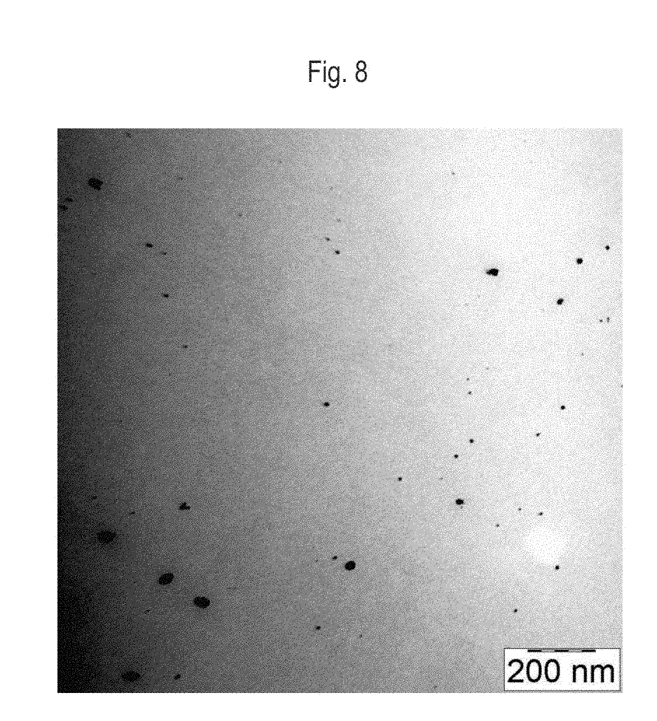

FIG. 8 shows, in exemplary manner, the TEM image of a polyester master batch containing 6,500 mg/kg silver. The dark spots in FIG. 8 show the homogeneous distribution and low agglomeration of the nanoparticles in the polyester.

The masterbatches were used, according to the prior art, appropriately diluted, for the production of synthetic fibres, e.g. made of polypropylene, polyester or polyamide.

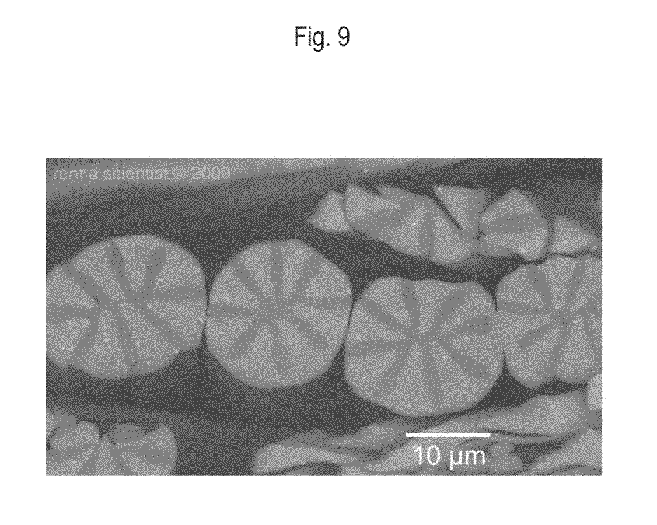

FIG. 9 shows several microfibre strands made of PET/PA containing 200 mg/kg nanosilver. The individual nanosilver particles (bright spots) in the segmented yarns are clearly visualised in FIG. 9. Typical silver contents for antimicrobial effects were in the range of 100 mg/kg to 300 mg/kg.

In addition to microfibres, monofilament and bi-component fibres for clothing, bedding, cloths, and technical textile materials as well as non-woven materials were produced. The silver-containing synthetic fibres can also be used in the form of staple fibres to equip other fibres (including natural fibres, such as, e.g., cotton). The silver contents used presently were higher as compared to direct equipment of the synthetic fibres in line with the dilution by other fibres.

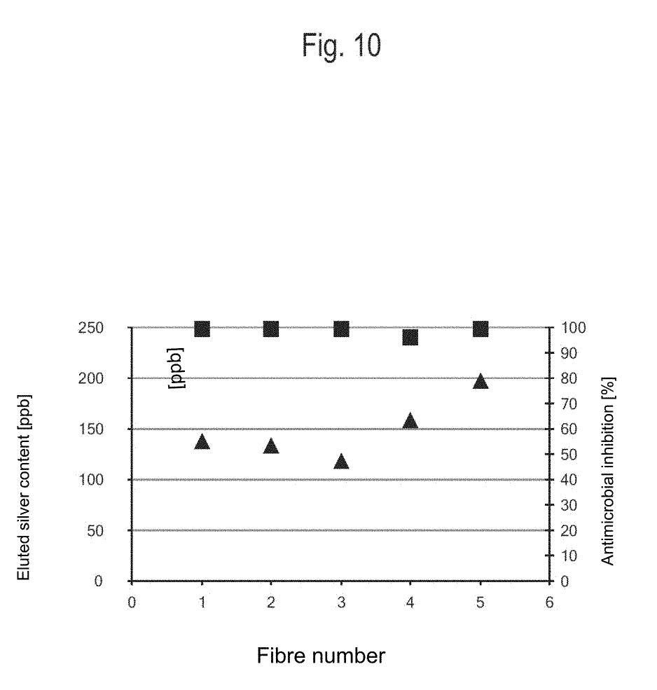

FIG. 10 shows the elution behaviour (▴) and the antimicrobial activity (▪) of different polyester microfibres. The silver content of the different fibres was in the range of 150 mg/kg to 195 mg/kg. The fibres were eluted in water for 3 h in each case. The eluted silver content was in the range of 120 μg/kg to 200 μg/kg water; the antimicrobial inhibition exceeded 96% in each case. Table 10 shows the results of the antimicrobial tests according to JIS 1902 on said 5 microfibres.

### Example 23

### Production of a Stable Dispersion of Silver Nanoparticles in Methylmethacrylate

The product from Example 6 or Example 7 was used to produce a formulation of silver nanoparticles in methylmethacrylate. For this purpose, 990 g methylmethacrylate (Merck, for synthesis) were placed in a 2 L beaker and stirred at room temperature on a magnetic stirrer. A pipette was used to add 1 g wetting agent (Evonic, Tego dispers 655). After the addition of 9.3 g of the product from Example 6, the colour of the solution changed to orange-brown. Swirling the glass allowed a thin film to be generated on the glass wall that was light-yellow in colour and clear and contained no visible particles. A total of 1,000 g of a dispersion with a silver content of 5,115 mg/kg were obtained. Particle size and particle distribution corresponded to the depiction in FIGS. 2 and 3.

### Example 24

### Production of a Stable Dispersion of Silver Nanoparticles in Methylmethacrylate

The product from Example 6 or Example 7 was used to produce a formulation of silver nanoparticles in methylmethacrylate. For this purpose, 980 g methylmethacrylate (Merck, p.a.) were placed in a 2 L beaker and stirred at room temperature on a magnetic stirrer. A pipette was used to add 2 g wetting agent (Evonic, Tego dispers 655). After the addition of 18.2 g of the product from Example 6, the colour of the solution changed to orange-brown. Swirling the glass allowed a thin film to be generated on the glass wall that was light-yellow in colour and clear and contained no visible particles. A total of 1,000 g of a dispersion with a silver content of 10,010 mg/kg were obtained. Particle size and particle distribution corresponded to the depiction in FIGS. 2 and 3.

### Example 25

### Production of a Stable Dispersion of Silver Nanoparticles in Methylmethacrylate

The product from Example 6 or Example 7 was used to produce a formulation of silver nanoparticles in methylmethacrylate. For this purpose, 897 g methylmethacrylate (Merck, for synthesis) were placed in a 2 L beaker and stirred at room temperature on a magnetic stirrer. A pipette was used to add 10 g wetting agent (Evonic, Tego dispers 655). After the addition of 92.7 g of the product from Example 6, the colour of the solution changed to orange-brown. Swirling the glass allowed a thin film to be generated on the glass wall that was light-yellow in colour and clear and contained no visible particles. A total of 1,000 g of a dispersion with a silver content of 50,985 mg/kg were obtained. Particle size and particle distribution corresponded to the depiction in FIGS. 2 and 3.

### Example 26

### Production of a PMMA-Based Antibacterial Carrier Material

The product from Example 1 was incorporated into a PMMA bead polymer at a concentration of 100 mg/kg to 10,000 mg/kg, each based on the finished product. It is preferred to incorporate this into the dry PMMA powder. Moreover, a pharmaceutically effective substance, such as gentamicin, as well as other additives, such as, for example, zirconium oxide as an X-ray contrast agent, can be added to the PMMA powder.

The PMMA powder was used, by injection moulding, to produce beads with an anti-microbial effect, for example having a diameter between 5 and 10 mm and a weight of 100 to 300 mg. This can be done without problem due to the heat resistance of the nanosilver dispersion up to 240° C. A bead of 200 mg can contain, for example, 4.5 g gentamicin and 20 mg zirconium oxide.

The beads can be anchored on a multi-filament surgical wire.

What is claimed:

1. A method for producing a dispersion containing silver nanoparticles, the method comprising:
providing a silver salt;
providing at least one stabiliser;
providing a reducing agent;
providing an organic polymerisable solvent;
producing an aqueous solution of silver salt, stabiliser, and reducing agent;
adding a base to the solution, so that silver nanoparticles are precipitated and dispersed;
adding an inorganic salt, the addition thereof resulting in the formation of two chemical phases including an aqueous phase and an organic phase, the organic phase comprising the organic polyermizable solvent, the at least one stabilizer, and the silver particles;
adding at least one wetting and dispersing additive; and
separating the aqueous phase from the organic phase.

2. The method of claim 1 wherein the at least one stabiliser is selected from the group consisting of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acid ester, polyoxypropylene-tri-alkyl acid ester, and mixtures thereof.

3. The method of claim 1 wherein the reducing agent comprises hydrazine hydrate.

4. The method of claim 1 wherein the organic polymerisable solvent comprises an acrylate.

5. The method of claim 1 wherein the base has a pK_{b} value in a range of −2 to 10.5.

6. The method of claim 1 wherein the base is selected from the group consisting of ammonia, potassium hydrogencarbonate, and sodium hydroxide.

7. The method of claim 1 wherein the inorganic salt comprises at least one element from Group IVA or Group VA of the periodic table as a component of the anion.

8. The method of claim 1 wherein the inorganic salt comprises nitrogen as a component of the anion.

9. The method of claim 1 wherein the wetting and dispersing additive comprises a non-ionic surfactant.

10. The method of claim 9, wherein the wetting and dispersing additive is selected from the group consistin gof an alkylphenolethoxylate, an amino-functional polyester, a phosphorus-containing substance, and mixtures thereof.

11. The method of claim 9, wherein the wetting and dispersing additive is an organically-modified phosphate, a phosphonate, a polyphosphorus compound, an alkylphosphonate, a phosphorus compound comprising mixed organic ligands, an oligomer or a polymer comprising phosphate-containing ligands.

12. The method of claim 1 wherein adding a base further comprises adding the base continuously such that the pH value of the formulation is between 0 and 6.

13. The method of claim 1 wherein adding a base further comprises adding the base continuously over a period of time in a range of 9 to 30 hours.

14. The method of claim 1 wherein separating the aqueous phase further comprises decanting the aqueous phase.

15. The method of claim 1 wherein elemental silver is formed by a reaction between the reducing agent and silver ions of the silver salt.

16. A dispersion containing silver nanoparticles producible by a method according to claim 1.

17. A composition of matter comprising a dispersion containing silver nanoparticles, the dispersion comprising:
silver nanoparticles;
at least one stabiliser; and
at least one wetting and dispersing additive comprising a non-ionic surfactant, wherein:
(a) the silver nanoparticles are dispersed in a liquid monomer, pre-polymer or polymer, and
(b) the stabiliser is selected from the group consisting of polyoxyethylene-mono-alkyl acid ester, polyoxypropylene-mono-alkyl acid ester, polyoxyethylene-di-alkyl acid ester, polyoxypropylene-di-alkyl acid ester, polyoxyethylene-tri-alkyl acid ester, and polyoxypropylene-tri-alkyl acid ester.

18. The composition of matter of claim 17 wherein the mean particle size of the silver nanoparticles is in a range between 5 to 50 nanometers.

19. The composition of matter of claim 17 wherein at least 90 percent of the silver nanoparticles are smaller than 50 nm.

20. The composition of matter of claim 17 wherein the silver nanoparticles have a spherical shape.

21. The composition of matter of claim 17 wherein the fraction of silver nanoparticles in the dispersion is between 0.5 and 5 percent by weight.

22. The composition of matter of claim 17 wherein the wetting and dispersing additive is selected from the group consisting of an organically-modified phosphate, a phosphonate, a polyphosphorus compound, an alkylphosphonate, a phosphorus compound comprising mixed organic ligands, an oligomer, and a polymer comprising phosphate-containing ligands.

23. The composition of matter of claim 17 wherein the polymer comprises an acrylate or an acrylate precursor.

24. The composition of matter of claim 17 wherein the silver nanoparticles are enveloped by the at least one stabiliser and the wetting and dispersing additive.

25. The composition of matter of claim 17 wherein the composition of matter is selected from the group consisting of a monomer, a coating solution, and an additive for polymer materials.

26. The composition of matter of claim 17 wherein the composition of matter is selected from the group consisting of bone cement, a coating agent for implants and medical instruments, and an antibacterial carrier material.

27. The composition of matter of claim 26, wherein the stabilisers are additionally selected from the group consisting of a polyoxyethylene-sorbitan-monolaurate, a polyoxyethylene-sorbitan-monopalmitate, a polyoxyethylene-sorbitan-monostearate, a polyoxyethylene-sorbitan-monooleate, a polyoxyethylene-sorbitan-tristearate, a polyoxyethylene-glyceryl-trioleate, a polyoxyethylene-glyceryl-monolaurate, a polyoxyethylene-glyceryl-monooleate, a polyoxyethylene-glyceryl-monostearate, a polyoxyethylene-glyceryl-monoricinoleate, a castor oil, a hydrogenated castor oil, a soy bean oil, and mixtures thereof.
